(19) United States
(12) Patent Application Publication
(10) Pub. No.: US 2010/0304501 A1
(43) Pub. Date: Dec. 2, 2010

(54) BIO LAB-ON-A-CHIP AND METHOD OF FABRICATING AND OPERATING THE SAME

(75) Inventors: Dae Sik Lee, Daejeon (KR); Rae Man Park, Daejeon (KR); Sung Lyul Maeng, Chungbuk (KR); Moon Youn Jung, Daejeon (KR); Seon Hee Park, Daejeon (KR); Hyun Chul Yoon, Gyeonggi-do (KR)

Correspondence Address: AMPACC Law Group, 3500 188th Street S.W., Suite 103, Lynnwood, WA, 98037, US

(73) Assignee: ELECTRONICS AND TELECOMMUNICAATIONS RESEARCH INSTI, DAEJEON (KR)

(21) Appl. No.: 12/740,348

(22) PCT Filed: Nov. 9, 2007
(86) PCT No.: PCT/KR07/05655

§ 371 Date: Aug. 18, 2010

(30) Foreign Application Priority Data
Nov. 9, 2007 (KR) 10-2007-0114116

(51) Int. Cl. G01N 33/543; G01N 30/96

(52) U.S. Cl. 436/518; 422/69

(57) ABSTRACT

Disclosed is a bio lab-on-a-chip. The bio lab-on-a-chip is provided on a piezoelectric thin film on a substrate, and includes a sensing unit to sense a bio signal and a fluidic control unit which controls a transfer of a microfluid adjacent to the sensing unit. Provided is also a method of fabricating the bio lab-on-a-chip. The method includes the steps of forming a piezoelectric thin film, forming a sensing unit to sense a bio signal of a microfluid on the piezoelectric thin film, and forming a fluidic control unit located adjacent to the sensing unit.

### TECHNICAL FIELD

Hi The present invention relates to a bio-micro electro mechanical system and a method of fabricating the same, and more particularly to a bio lab-on-a-chip and methods of fabricating and operating the same.

The present invention has been derived from research undertaken as a part of IT R & D program of the Ministry of Information and Communication and Institution of Information Technology Association (MIC/IITA) [2006-S-007-02], Ubiquitous health monitoring module and system development.

### BACKGROUND ART

Generally, in the field of bio-micro electro mechanical systems (Bio-MEMS), to perform processes such as early diagnosis of diseases and/or chemical analysis on a small chip, a microfluidic control capable of transferring, stopping, mixing, and reacting an ultra-low volume fluid, and an integration of sensors capable of sensing bio markers, for example, protein, deoxyribonucleic acid (DNA), related to diseases must be required.

In the Bio-MEMS field, particularly in the field of chemical analysis and/or early diagnosis of diseases, researches on miniaturization, low cost, integration, automation, and real-time diagnosis are actively under development. Since most of the generic reagents are generally expensive, a reproducible and contamination-free chemical analysis must be performed using a minimum volume of a bio sample. Accordingly, low-price microfluidic control systems have attracted a special attention.

However, a conventional microfluidic control system is a mere continuous control system to control a fluid flow by changing a flow rate, preventing a fluid flow and/or causing a reaction by means of intersecting different fluid flows. In addition, a detection sensor to detect a bio signal of a conventional fluid sample is just a system such as enzyme-linked immunosorbent assay (ELISA) which uses reactions within a container like a tube, and to utilize reactions in a continuous flow as a fluid form like an electrochemical luminescence, fluorescent luminescence and/or surface plasmon resonance (SPR).

In particular, in order to be used in a chemical analysis and an early diagnosis of diseases in a farm of lab-on-a-chip, a microfluidic control system which can transfer, stop, mix, and react a fluid rapidly and exactly while consuming an extremely small volume of a sample, and a detection sensor which can immobilize and sense an antigen like a bio marker must be combined.

So far, to transfer a fluid at a liquid-drop level, a separate microactuator has been used as a microfluidic control system to enhance and stop the fluidic mobility. This type of the microfluidic control system transfers, stops, mixes and reacts a fluid at a liquid-drop level using a pressure difference caused by an actuator, for example, piezoelectric, thermopneumatic, and a microfluidic control system and the actuator are driven individually within the system.

On the contrary, a microfluidic control system has been presented, which can transfer, stop, mix, and react a fluid only using the capillary force caused in a micro channel and the geometry of the channel without a separate actuator. Since this type of the microfluidic control system has a continuous flow of a fluid consisting of a bio sample, the system has the disadvantage that the larger amounts of the bio sample and the expensive reagents mixed therewith should be consumed to sense a bio marker substantially. The system also has the disadvantage that a separate device should be required to maintain the dispersion of a target bio material such as protein, cell, and DNA, in the fluid.

Until now, most of the sensing of bio markers have been performed in a continuous flow of a fluid. The sensing of bio markers using surface acoustic wave (SAW) also follows this pattern. A bio sensor was also presented to quantify various analysis materials using a bulk substrate made of quartz. The quartz bulk substrates in the bio sensor are disadvantageous in that they are expensive and hardly applied to a general semiconductor manufacturing process based on typical silicon substrates. In addition, when a microfluidic control system and a detection sensor are fabricated on a single chip, peripheral signal processing circuits, for example, amplifier circuit, analog/digital converter, cannot be integrally formed.

### DISCLOSURE OF INVENTION

### Technical Problem

The present invention provides a bio lab-on-a-chip, which is capable of transferring, reacting, and sensing a microfluid on a single chip while minimizing the amount of a sample used.

The present invention also provides a method of fabricating a bio lab-on-a-chip capable of transferring, reacting, and sensing a microfluid on a single chip while minimizing the amount of a sample used.

The present invention also provides a method of operating a bio lab-on-a-chip capable of transferring, reacting, and sensing a microfluid on a single chip while minimizing the amount of a sample used.

Technical Solution

Embodiments of the present invention provide bio lab-on-a-chips may include: a substrate; a piezoelectric thin film on the substrate; a sensing unit provided on the piezoelectric thin film, and sensing a bio signal of a microfluid; and a fluidic control unit adjacent to the sensing unit, and controlling a transfer of the microfluid.

In some embodiments, the lab-on-a-chip may further include a microfluidic channel disposed on the piezoelectric thin film between the sensing unit and the fluidic control unit. The microfluidic channel may include a hydrophobic material. The hydrophobic material may include at least one material selected from a silane compound, a carbon nanotube, and diamond like carbons.

In other embodiments, the substrate may include at least one selected from silicon, glass, plastic, metal, and a combination thereof.

In still other embodiments, the piezoelectric thin film may have a thickness in the range of about 0.1 μm to about 10 μm. The piezoelectric thin film may include at least one selected from ZnO, AlN, LiNbO₃, LiTaO₃, quartz, polymer, and a combination thereof.

In even other embodiments, the bio lab-on-a-chip may further include antibodies provided on the sensing unit. The antibodies may include a self-assembling monolayer (SAM) or protein.

In yet other embodiments, the bio lab-on-a-chip may further include a pair of interdigitated transducers disposed adjacent to the sensing unit in a vertical direction to a virtual line connecting the fluidic control unit and the sensing unit, wherein the sensing unit is positioned between the pair of interdigitated transducers.

In further embodiments, the pair of interdigitated transducers may include a selected interdigitated transducer sending a surface acoustic wave (SAW) to the sensing unit and an unselected interdigitated transducer converting a modulated SAW by the sensing unit into an electrical signal.

In still further embodiments, the fluidic control unit may be an interdigitated transducer which provides a SAW in a direction to the sensing unit.

In even further embodiments, the bio lab-on-a-chip may further include a dam portion which surrounds the sensing unit and the microfluidic channel. The dam portion may include a photosensitive polymer.

In other embodiments of the present invention, to solve the other technical problems described above, methods for fabricating a bio lab-on-a-chip may include: forming a piezoelectric thin film on a substrate; forming a sensing unit on the piezoelectric thin film, the sensing unit sensing a bio signal of a microfluid: and forming a fluidic control unit adjacent to the sensing unit, the fluidic control unit controlling a transfer of the microfluid.

In some embodiments, the piezoelectric thin film may be formed to have a thickness in the range of about 0.1 μm to about 10 μm.

In other embodiments, the forming of the piezoelectric thin film may include the steps of depositing a piezoelectric material on the substrate and heat-treating the deposited piezoelectric material. The piezoelectric material may include at least one selected from ZnO, AlN, LiNbO₃, LiTaO₃, quartz, polymer, and a combination thereof.

In still other embodiments, the depositing of the piezoelectric material may include at least one method selected from a reactive sputtering method, a chemical vapor deposition (CVD) method, a molecular beam epitaxy method, an atomic layer deposition (ALD) method, and a combination thereof.

In even other embodiments, the fluidic control unit may have a form of an interdigitated transducer.

In yet other embodiments, the forming of the fluidic control unit may be performed prior to the forming of the piezoelectric thin film.

In further embodiments, the sensing unit and the fluidic control unit may be formed simultaneously.

In still further embodiments, the forming of the sensing unit and the fluidic control unit simultaneously may include: forming a photoresist pattern which exposes a sensing unit region and a fluidic control unit region on the piezoelectric thin film; forming a conductive metal film on the photoresist pattern and on the piezoelectric thin film exposed by the photoresist pattern; and removing the photoresist pattern and the conductive metal film on the photoresist pattern by a lift-off process.

In even further embodiments, the forming of a pair of interdigitated transducers disposed adjacent to the sensor may be further included in a vertical direction to a virtual line connecting the fluidic controller and the sensor, wherein the sensor is positioned between the pair of interdigitated transducers.

In yet further embodiments, the pair of interdigitated transducers may be formed simultaneously with the fluidic control unit.

In some embodiments, the pair of interdigitated transducers may be formed simultaneously with the sensing unit and the fluidic control unit.

In other embodiments, the forming of antibodies on the sensing unit may be further included. The antibodies may include a self-assembling monolayer (SAM) or protein.

In still other embodiments, the forming of a dam portion which surrounds the sensing unit and the microfluidic channel may be further included. The dam portion may be formed of a photosensitive polymer.

In still other embodiments of the present invention, to solve the other technical problems described above, methods for operating a bio lab-on-a-chip may include: providing a microfluid to a region between a sensing unit and a fluidic control unit adjacent to each other on a substrate having a piezoelectric material; transferring the microfluid to the sensing unit using a surface acoustic wave (SAW) generated by driving the fluidic control unit; and sensing a bio signal of the microfluid at the sensing unit.

In some embodiments, the fluidic control unit may be an interdigitated transducer for fluid control, which provides the SAW.

In other embodiments, the microfluid may be a liquid drop of nanoliters in volume.

In still other embodiments, the microfluid may include one of an optical marker material and a radioactive marker material.

In even other embodiments, the sensing of the bio signal of the microfluid may include sensing a reaction between antibodies provided on the sensing unit and the microfluid as an optical signal or a radioactive signal.

In even other embodiments, the sensing of the bio signal of the microfluid may include sensing a reaction between antibodies provided on the sensing unit and the microfluid as an electrical signal. The sensing of the electrical signal may use at least one interdigitated transducer disposed adjacent to the sensing unit, and measure a resonance frequency modulated as an SAW generated from the interdigitated transducer passes through the sensing unit.

In yet other embodiments, a variation of the resonance frequency of the SAW may be proportional to the amount of a reaction between the antibodies and the microfluid.

In further embodiments, the interdigitated transducer may include a first detection interdigitated transducer sending the SAW to the sensing unit and a second detection interdigitated transducer detecting the modulated SAW at the sensing unit.

In even other embodiments of the present invention, methods for operating a bio lab-on-a-chip may include: providing a detection sensor on a piezoelectric material, the detection sensor sensing a bio signal of a microfluid; providing a surface acoustic wave (SAW) to the detection sensor; and measuring a resonance frequency of a modulated SAW by a reaction between the detection sensor and the microfluid, wherein a variation of the resonance frequency of the SAW may be proportional to the amount of the reaction between the detection sensor and the microfluid.

In some embodiments, the providing of the SAW may include using at least one interdigitated transducer adjacent to the detection sensor.

In other embodiments, the interdigitated transducer may include: a first detection interdigitated transducer sending the SAW to the detection sensor; and a second detection interdigitated transducer detecting the modulated SAW at the detection sensor.

### ADVANTAGEOUS EFFECTS

As described in detail above, according to the present invention, it is possible to transfer, stop, react, and sense a microfluid in the form of a micro-sized drop solution on a single chip. Accordingly, a bio lab-on-a-chip may be provided to reduce analysis cost by minimizing the consumption of a bio sample and reagents. Further, since all the processes of a chemical analysis are performed on a single chip, a bio lab-on-a-chip may be provided for a rapid and exact analysis. In addition, a bio lab-on-a-chip may be provided to reduce fabrication cost by replacing an expensive bulk substrate with a piezoelectric thin film. Additionally, a signal-processing unit can be integrated on a single chip using a general semiconductor manufacturing process. Therefore, this can be also applicable to various bio lab-on-a-chip fields such as a protein lab-on-a-chip, polymerase chain reaction (PCR), DNA lab-on-a-chip and a micro biological/chemical reactor.

### BRIEF DESCRIPTION OF THE DRAWINGS

The accompanying figures are included to provide a further understanding of the present invention, and are incorporated in and constitute a part of this specification. The drawings illustrate exemplary embodiments of the present invention and, together with the description, serve to explain principles of the present invention. In the figures:

FIG. 1 is a perspective view of a bio lab-on-a-chip according to an embodiment of the present invention;

FIGS. 2 through 5 are conceptual cross-sectional views illustrating reactions in a sensing unit of a bio lab-on-a-chip according to an embodiment of the present invention;

FIG. 6 is a scanning electron microscope image illustrating a piezoelectric thin film of a bio lab-on-a-chip according to an embodiment of the present invention;

FIG. 7 is a graph illustrating a crystalline state of a piezoelectric thin film of a bio lab-on-a-chip according to an embodiment of the present inventions;

FIG. 8 is a graph illustrating a resonance characteristic of a piezoelectric thin film of a bio lab-on-a-chip according to an embodiment of the present invention;

FIGS. 9 through 12 are conceptual cross-sectional views illustrating a sensing unit of a bio lab-on-a-chip according to an embodiment of the present invention;

FIG. 13 is a graph illustrating transitions of a resonance frequency and an amplitude of a bio lab-on-a-chip according to an embodiment of the present invention;

FIG. 14 is a graph illustrating a transition degree of a resonance frequency depending on the amount of antigens of a bio lab-on-a-chip according to an embodiment of the present invention;

FIGS. 15 through 24 are cross-sectional views taken along line I-I′ of FIG. 1, illustrating a method of fabricating a bio lab-on-a-chip according to an embodiment of the present invention; and

FIGS. 25 through 31 are cross-sectional views taken along line I-I′ of FIG. 1, illustrating a method of fabricating a bio lab-on-a-chip according to another embodiment of the present invention.

### BEST MODE FOR CARRYING OUT THE INVENTION

Preferred embodiments of the present invention will be described below in more detail with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art. Additionally, because the reference numerals have been used to clarify a preferred embodiment, their sequences in description may not necessarily be limited to a numerical order. In the figures, the dimensions of layers and regions are exaggerated for clarity of illustration. It will also be understood that when a layer (or film) is referred to as being ‘on’ another layer or substrate, it can be directly on the other layer or substrate, or intervening layers may also be present. Further, it will be understood that when a layer is referred to as being ‘under’ another layer, it can be directly under, and one or more intervening layers may also be present. In addition, it will also be understood that when a layer is referred to as being ‘between’ two layers, it can be the only layer between the two layers, or one or more intervening layers may also be present.

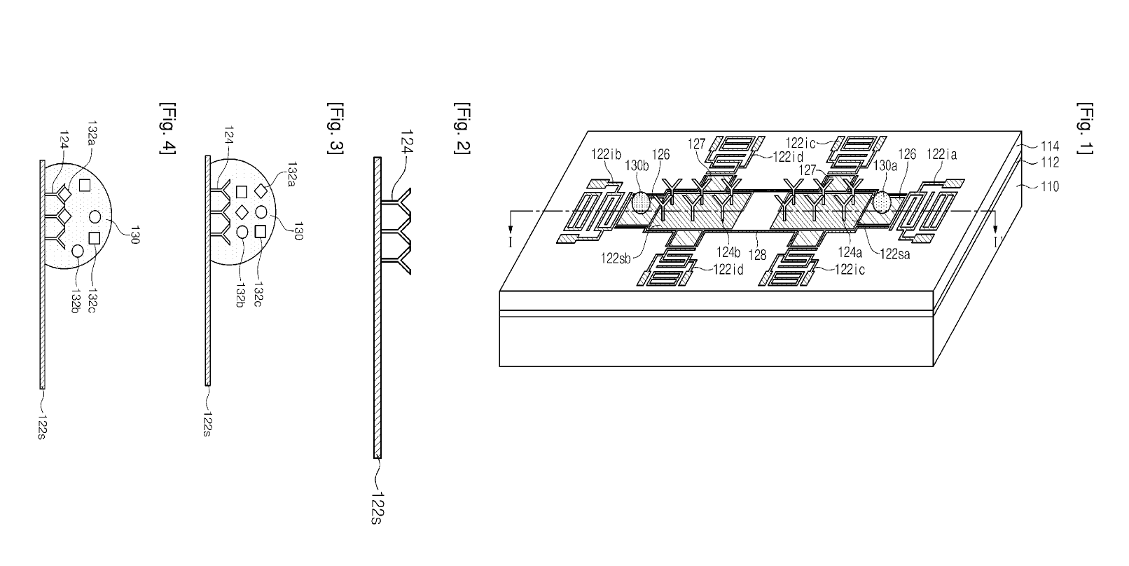

FIG. 1 is a perspective view of a bio lab-on-a-chip according to an embodiment of the present invention.

Referring to FIG. 1, a bio lab-on-a-chip may include a substrate 110, a piezoelectric thin film 114, sensors 122sa and 122sb, and fluidic controllers 122ia and 122ib. The bio lab-on-a-chip may further include a microfluidic channel 126 disposed between the sensors 122sa and 122sb and the fluidic controllers 122ia and 122ib.

The substrate 110 may include at least one selected from silicon (Si), glass, plastic, metal, and a combination thereof. Preferably, the substrate 110 may be a silicon substrate.

The piezoelectric thin film 114 may be provided on the substrate 110. The piezoelectric thin film 114 may have a thickness in the range of about 0.1 μm to about 10 μm. Preferably, the piezoelectric thin film 114 may have a thickness in the range of about 0.5 μm to about 10 μm. The piezoelectric thin film 114 may include at least one selected from ZnO, AlN, LiNbO₃, LiTaO₃, quartz, polymer, and a combination thereof. Preferably, the piezoelectric thin film 114 may be a deposited film having a thickness of about 5.5 μm of ZnO.

A silicon oxide (SiO₂) film 112 may be disposed between the substrate 110 and the piezoelectric thin film 114. The SiO₂ film 112 may be provided for minimizing the loss of surface acoustic wave (SAW), which should propagate along the piezoelectric thin film 114, by preventing the SAW from propagating to the substrate 110.

The sensors 122sa and 122sb may be provided on the piezoelectric thin film 114. The sensors 122sa and 122sb may be a conductive metal film. The conductive metal film may include at least one selected from gold (Au), silver (Ag), aluminum (Al), platinum (Pt), tungsten (W), nickel (Ni), copper (Cu), and a combination thereof. Preferably, the sensors 122sa and 122sb may be an Au-deposited film.

As illustrated in FIG. 1, the sensors 122sa and 122sb according to the embodiment of the present invention may include a first sensor 122sa and a second sensor 122sb. Since a bio lab-on-a-chip includes a reference sensor for calibration of the bio lab-on-a-chip and a sample sensor for analysis of bio samples, a pre-calibration may not be required for the bio lab-on-a-chip. In addition, if the bio lab-on-a-chip is pre-calibrated, a simultaneous analysis of two bio samples may be performed.

Antibodies 124a and 124b may be further provided on the sensors 122sa and 122sb.

The antibodies 124a and 124b may include a self-assembling monolayer (SAM) or protein. Antigens in microfluids 130a and 130b through an immunological reaction such as an antigen-antibody reaction may be adhered to the sensors 122sa and 122sb by the antibodies 124a and 124b.

The fluidic controllers 122ia and 1221b may be an interdigitated transducer (IDT) which provides the SAW in the direction of the sensors 122sa and 122sb. The fluidic controllers 122ia and 122ib may be a conductive metal film. The conductive metal film may include at least one selected from Au, Ag, Al, Pt, W, Ni, Cu, and a combination thereof. Preferably, the fluidic controllers 122ia and 1221b may be an Au-deposited film the same as the sensors 122sa and 122sb.

The microfluidic channel 126 may be provided on the piezoelectric thin film 114 between the sensors 122sa and 122sb and the fluidic controllers 122ia and 1221b. The microfluidic channel 126 may include a hydrophobic material. The hydrophobic material may include at least one selected from a silane compound, a carbon nanotube (CNT), and diamond like carbon (DLC). Accordingly, the microfluids 130a and 130b in the form of a liquid drop may be transferred to the sensors 122sa and 122sb through the microfluidic channel 126 while maintaining their forms.

Sensing interdigitated transducers 122ic and 122id may be further provided adjacent to the sensors 122sa and 122sb in a vertical direction to a virtual line connecting the fluidic controllers 122ia and 122ib to the sensors 122sa and 122sb. The sensing interdigitated transducers 122ic and 122id may be a conductive metal film. The conductive metal film may include at least one selected from Au, Ag, Al, Pt, W, Ni, Cu, and a combination thereof. Preferably, the sensing interdigitated transducers 122ic and 122id may be an Au-deposited film the same as the sensors 122sa and 122sb.

The sensing interdigitated transducers 122ic and 122id may include a pair of interdigitated transducers between which the sensors 122sa and 122sb may be disposed. The sensing interdigitated transducers 122ic and 122id may include a first interdigitated transducer for sensing which sends the SAW to the sensors 122sa and 122sb and a second interdigitated transducer for sensing which detects the SAW modulated by the sensors 122sa and 122sb. The first interdigitated transducer and the second interdigitated transducer may face each other with the sensors 122sa and 122sb interposed therebetween. In addition, a microfluidic channel 127 may be provided on the piezoelectric thin film 114 between the sensors 122sa and 122sb and the sensing interdigitated transducers 122ic and 122id. The microfluidic channel 127 may be provided for easily removing the microfluids 130a and 130b which have completed reactions with the antibodies 124a and 124b in the sensors 122sa and 122sb, using the SAWs generated from the sensing interdigitated transducers 122ic and 122id.

Since the fluidic controllers 122ia and 1221b and the sensing interdigitated transducers 122ic and 122id have a form of an interdigitated transducer, it may be preferred for them to be formed simultaneously in the same process. Unlike FIG. 1, the fluidic controllers 122ia and 122ib and the sensing interdigitated transducers 122ic and 122id may be provided below the piezoelectric thin film 114.

A dam portion 128 which surrounds the sensors 122sa and 122sb and the microfluidic channels 126 and 127 may be further included. The dam portion 128 may include a photosensitive polymer. Accordingly, the microfluidics 130a and 130b in the form of a liquid drop may be stably transferred to the sensors 122sa and 122sb through the microfluidic channel 126 without deviating outside.

An example of a method of operating a bio lab-on-a-chip in the above may be as follows.

The microfluids 130a and 130b may be provided in the microfluidic channel 126 between the fluidic controllers 122ia and 1221b and the sensors 122sa and 122sb disposed adjacent to each other on the substrate 110 provided with the piezoelectric thin film 114. The microfluids 130a and 130b may be liquid drops of nanoliters (nl) in volume. The microfluids 130a and 130b may also include an optical marker material or a radioactive marker material.

The SAW directed to the sensors 122sa and 122sb may be produced by driving the fluidic controllers 122ia and 122ib. The microfluids 130a and 130b may be moved toward the sensors 122sa and 122sb, by the SAW produced by driving the fluidic controllers 122ia and 122ib. If the driving of the fluidic controllers 122ia and 122ib would stop, the microfluids 130a and 130b may be stopped on the sensors 122sa and 122sb.

The microfluids 130a and 130b moved to the sensors 122sa and 122sb react with the antibodies 124a and 124b provided on the sensors 122sa and 122sb. Antigens included in the microfluids 130a and 130b may cause an antigen-antibody reaction with the antibodies 124a and 124b, and then adhere to the sensors 122sa and 122sb.

A bio signal may be sensed from the antigens adhering to the sensors 122sa and 122sb. The sensing of the bio signal may be to measure an optical signal or a radioactive signal with respect to the antigens binding with the optical marker material or the radioactive marker material. On the contrary, the sensing of the bio signal may be to measure a resonance frequency modulated as the SAW generated from the sensing interdigitated transducers 122ic and 122id passes through the sensors 122sa and 122sb to which the antigens are adhering. For example, as the SAW generated from the first interdigitated transducer passes through the sensors 122sa and 122sb, the resonance frequency thereof may be modulated and detect the modulated SAW in the second interdigitated transducer.

Another example of the method of operating a bio lab-on-a-chip as above may be as follows.

Each of the microfluidics 130a and 130b may be provided in each of the microfluidic channels 126 disposed between the fluidic controllers 122ia and 122ib and the sensors 122sa and 122sb disposed adjacent to each other on the substrate 110 with the piezoelectric thin film 114 formed. The microfluids 130a and 130b may be liquid drops of nanoliters in volume. The microfluids 130a and 130b may also include an optical marker material or a radioactive marker material.

Each of the SAWs directed to the sensors 122sa and 122sb may be produced by driving the fluidic controllers 122ia and 1221b. The fluidic controllers 122ia and 122ib may also include a first fluidic controller 122ia and a second fluidic controller 122ib. The microfluids 130a and 130b may be moved toward each of the sensors 122sa and 122sb, by the SAWs produced by driving the fluidic controllers 122ia and 122ib. The sensors 122sa and 122sb may include a first sensor 122sa and a second sensor 122sb. If the driving of the fluidic controllers 122ia and 122ib would stop, the microfluids 130a and 130b may be stopped on each of the sensors 122sa and 122sb.

The microfluids 130a and 130b moved to the sensors 122sa and 122sb respectively may react with a first antibodies 124a and a second antibodies 124b respectively provided on the sensors 122sa and 122sb. Each of antigens included in the microfluids 130a and 130b respectively may cause an antigen-antibody reaction with each of the first antibodies 124a and the second antibodies 124b, and then adhere to the sensors 122sa and 122sb, respectively.

Each of bio signals may be sensed from each of the antigens adhering to each of the sensors 122sa and 122sb. The sensing of the bio signals may be to measure an optical signal or a radioactive signal with respect to each of the antigens binding with the optical marker material or the radioactive marker material. On the contrary, the sensing of the bio signals may be to measure resonance frequencies modulated as the SAWs generated from the sensing interdigitated transducers 122ic and 122id passes through the sensors 122sa and 122sb to which the antigens are adhering. For example, as the SAWs generated in the first interdigitated transducers pass through each of the sensors 122sa and 122sb, each of the resonance frequencies thereof may be modulated and detect the modulated SAW in the second interdigitated transducers.

The first sensor 122sa and the second sensor 122sb may be a standard sensor and a sample sensor, respectively. Since a bio lab-on-a-chip includes a standard sensor for calibration of the bio lab-on-a-chip and a sample sensor for analysis of bio samples simultaneously, a pre-calibration may not be required for the bio lab-on-a-chip. In addition, since a background noise of the bio lab-on-a-chip may be removed by the standard sensor, an exact analysis may be made for the bio sample. The first microfluid 130a provided to the standard sensor, i.e., the first sensor 122sa may be a standard sample. Also, a microfluid may be provided only to the sample sensor, i.e., the second sensor 122sb, not to the standard sensor.

In addition, the first sensor 122sa and the second sensor 122sb may be a first sample sensor and a second sample sensor, respectively. If a bio lab-on-a-chip is pre-calibrated, a simultaneous analysis would be possible for the two bio samples in the first sample sensor and the second sample sensor, respectively.

Since the bio lab-on-a-chip as described above transfer, stop, react, and sense a microfluid as a form of a nanoliter volume drop solution using a piezoelectric thin film, all the processes of a chemical analysis may be also performed on a single chip while using a minimum volume of a sample. Accordingly, the costs of analysis may be lowered simultaneously with the reduced fabricating costs of the bio lab-on-a-chip.

FIGS. 2 through 5 are conceptual cross-sectional views illustrating reactions in a sensing unit of a bio lab-on-a-chip according to an embodiment of the present invention.

Referring to FIGS. 2 and 3, antibodies 124 may be provided on a sensor 122s. The antibodies 124 may include a self-assembling monolayer (SAM) or protein.

A microfluid 130 may be transferred to the sensor 122s by an SAW produced from a fluidic controller (See 122ia or 122ib in FIG. 1). The microfluid 130 may be a nanoliter volume liquid drop including various kinds of antigens 132a, 132b and 132c. The microfluid 130 may also include an optical marker material or a radioactive marker material.

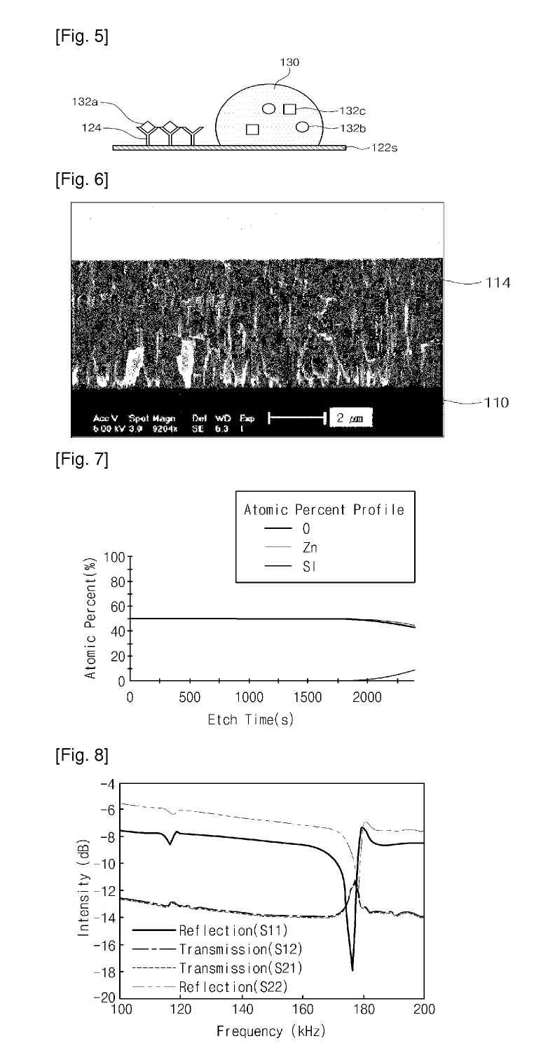

Referring to FIGS. 4 and 5, only the specific antigens 132a of the microfluid 130 may cause an antigen-antibody reaction with and bind to the antibodies 124. Accordingly, the specific antigens 132a in the microfluid 130 may adhere to the sensor 122s.

A bio signal may be sensed from the antigens 132a adhering to the sensor 122s. The sensing of the bio signal may be to measure an optical signal or a radioactive signal with respect to the antigens 132a binding with the optical marker material or the radioactive material included in the microfluid 130. On the contrary, the sensing of the bio signal may be to measure a resonance frequency modulated as the SAW generated from the sensing interdigitated transducers (See 122ic and 122id in FIG. 1) passes through the sensor 122s to which the specific antigens 132a are adhering.

The microfluid 130, including the antigens 132b and 132c which do not cause the antigen-antibody reaction with the antibodies 124 provided on the sensor 122s, may be removed by the SAW produced from the fluidic controller and the sensing interdigitated transducers.

FIG. 6 is a scanning electron microscope image illustrating a piezoelectric thin film of a bio lab-on-a-chip according to an embodiment of the present invention, and FIG. 7 is a graph illustrating a crystalline state of a piezoelectric thin film of a bio lab-on-a-chip according to an embodiment of the present inventions.

Referring to FIG. 6, an image of a piezoelectric thin film 114 deposited on a substrate 110 was taken using a scanning electron microscope (SEM). The substrate 110 may be a silicon substrate, and the piezoelectric thin film 114 may be a film which is heat-treated at about 400° C. under N₂ atmosphere for 10 minutes after ZnO is deposited in a thickness of about 5.5 μm by a reactive sputtering method. As illustrated in FIG. 6, it is understood that a thin film of ZnO may be also grown as a pillar-shaped structure on a silicon substrate.

Referring to FIG. 7, a graph shows an analysis of the piezoelectric thin film 114 on the substrate 110 using X-ray photoelectron spectroscopy (XPS). It is understood that the stoichiometrical atomic composition ratio of zinc to oxygen is 1:1 in a ZnO thin film in a depth direction of the piezoelectric thin film 114. This crystallographical composition ratio is estimated with reference to the value of ZnO in a bulk substrate.

It is understood that the piezoelectric thin film 114 may be grown well as a wurtzite structure in the crystal direction (0 0 2) using X-ray diffractometry (XRD) (not shown). In addition, it can be confirmed that the grain size of the piezoelectric thin film 114 is about 20 nm through the Scherr equation.

As a result, a piezoelectric thin film may be formed on a general-purpose silicon substrate, and it was confirmed that this piezoelectric thin film has a good crystallinity like a bulk substrate.

FIG. 8 is a graph illustrating a resonance characteristic of a piezoelectric thin film of a bio lab-on-a-chip according to an embodiment of the present invention.

Referring to FIG. 8, resulting values of scattering parameters (S-parameters) measured using a vector network analyzer (VNA) are illustrated to know the resonance characteristic of a piezoelectric thin film of a bio lab-on-a-chip. The S-parameters are the most widely used resulting values of circuits in a radio frequency (RF). S11 and S22 in the S-parameters are the values indicating the ratio of the RF intensity inputted to an input port to the reflected RF intensity outputted from the input port, while S12 and S21 are the values indicating the ratio of the inputted RF intensity to the input port to outputted RF intensity from an output port.

S11 and S22 are the values measured for the reflection characteristics of a piezoelectric thin film using a pair of interdigitated transducers used as input and output ports. S12 and S21 are the values measured for the transmission characteristic of the piezoelectric thin film. As illustrated in FIG. 8, it is understood that a ZnO piezoelectric thin film according to embodiments of the present invention has resonance characteristics in the specific frequencies of about 175 MHz (Sezawa mode) and about 120 MHz (Rayleigh mode).

The resonance is also found to occur in the piezoelectric thin film as in a bulk substrate. Accordingly, transferring, reacting, and sensing of a microfluid may be performed by the resonance characteristic of the piezoelectric thin film by a surface acoustic wave (SAW). The transferring, reacting, and sensing of the microfluid may be controlled by the sequence of the RF applied to a fluidic controller and/or first and second sensing interdigitated transducers, and the intensity of the RF energy applied respectively. It could be confirmed that when RF energy of about 44 V was applied to the fluidic controller as a form of an interdigitated transducer at about 175 MHz resonance frequency, about 200 nl size drop solution propagated at about 20 mm/s.

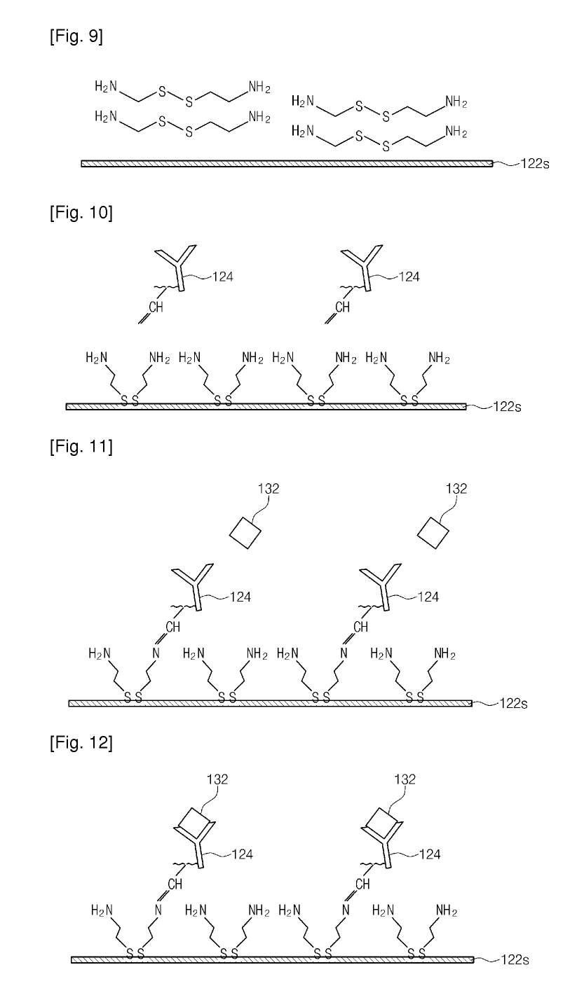

FIGS. 9 through 12 are conceptual cross-sectional views illustrating a sensing unit of a bio lab-on-a-chip according to an embodiment of the present invention. It is to describe an immune reaction for analysis of a prostate-specific antigen (PSA) protein included in a bio sample as an example.

Referring to FIGS. 9 and 10, cystamines (NH₂—CH₂—CH₂—S—S—CH₂—CH₂—NH) may be provided on a sensor 122s in a bio lab-on-a-chip. The sensor 122s may be an Au-deposited film. A cystamine self-assembling monolayer (SAM) may be formed on the sensor 122s by the covalent bonds generated between the S atoms included in the cystamines and a surface of the sensor 122s. Anti-PSA antibodies 124 are provided on the sensor 122s covered with the cystamine SAM.

Referring to FIGS. 11 and 12, the anti-PSA antibodies 124 may be immobilized to the sensor 122s by the covalent bonds generated between N atoms included in the cystamines of the cystamine SAM and C atoms included in the anti-PSA antibodies. At this time, H atoms binding to the N atoms included in the cystamines which are covalently bound to the C atoms in the anti-PSA antibodies 124, may be removed and exhausted during the covalent bonds.

PSAs 132 are provided on the sensor 122s immobilized with anti-PSA antibodies 124. Immuno-complexes in which PSAs 132 are binding to anti-PSA antibodies 124 through immune reactions may be formed. These immuno-complexes may be maintained while adhering to the sensor 122s by the cystamine SAM.

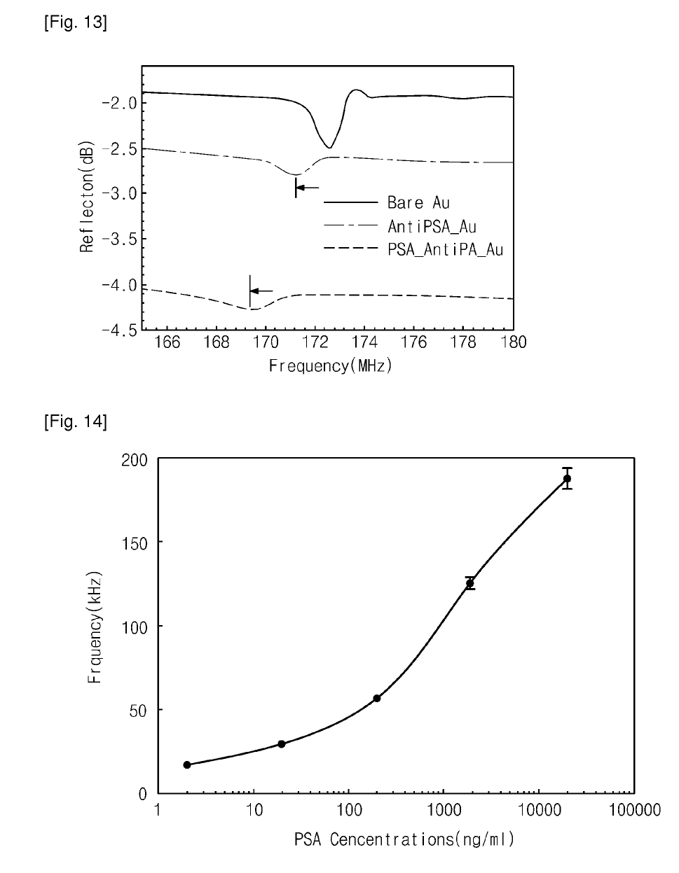

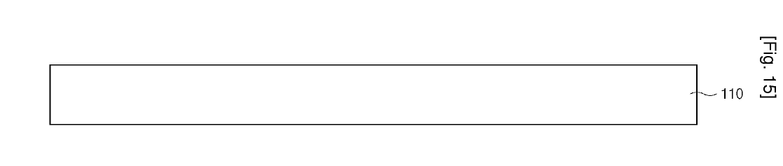

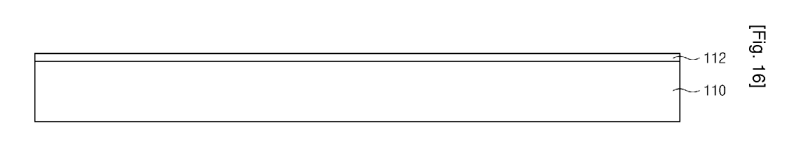

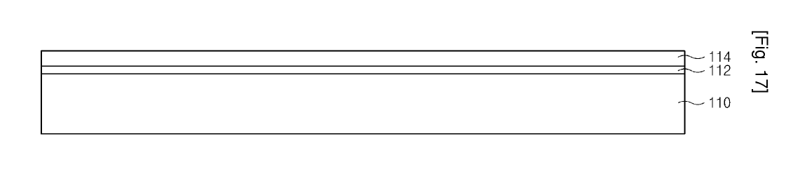

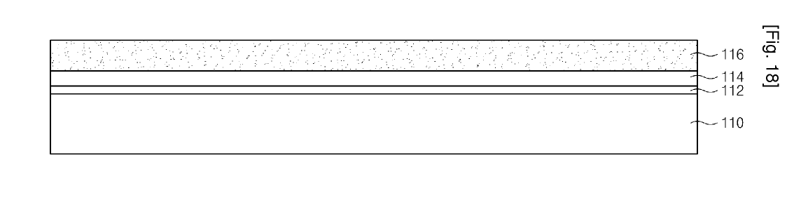

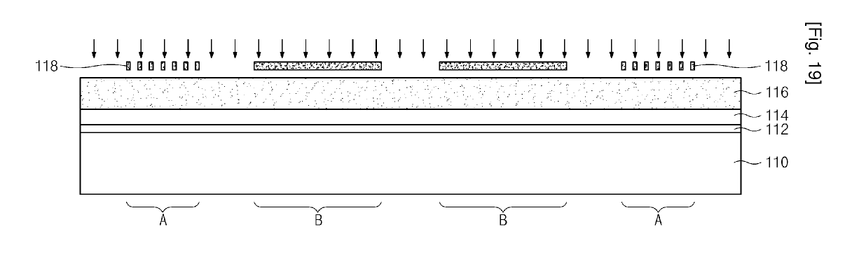

FIG. 13 is a graph illustrating transitions of a resonance frequency and an amplitude of a bio lab-on-a-chip according to an embodiment of the present invention.

Referring to FIG. 13, as anti-PSA antibodies (See 124 in FIG. 10) and PSAs (See 132 in FIG. 11) are sequentially adhering to an Au-deposited sensor (See 122s in FIG. 9) located between sensing interdigitated transducers (See 122ic and/or 122id in FIG. 1), the resonance frequency and the intensity thereof are measured in a graph.

It can be confirmed that as the anti-PSA antibodies and the PSAs are sequentially adhering to the Au-deposited sensor, the resonance frequency and the intensity thereof become lowered.

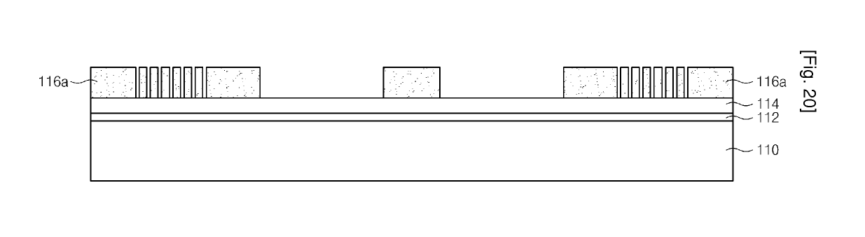

FIG. 14 is a graph illustrating a transition degree of a resonance frequency depending on the amount of antigens of a bio lab-on-a-chip according to an embodiment of the present invention.

FIG. 14 illustrates the resonance frequencies depending on the amounts of PSAs reacting with and adhering to anti-PSA antibodies provided on a sensor of a bio lab-on-a-chip.

It is understood that as the amounts of the PSAs adhering to the sensor change in the range from about 2 ng/ml to about 20,000 ng/ml, the resonance frequencies shift. The variation amounts of the resonance frequency tend to be exponentially proportional to the amounts of the PSAs adhering to the sensor. That is, a quantitative measurement of antigens adhering to the sensor may be possible.

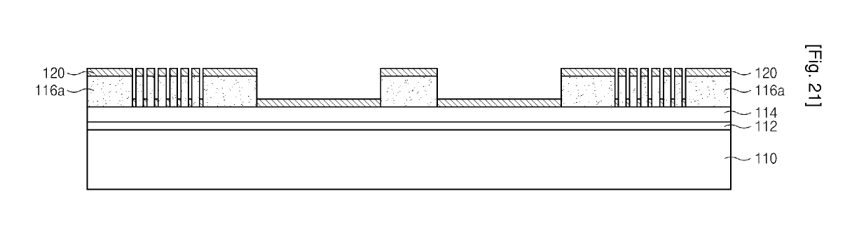

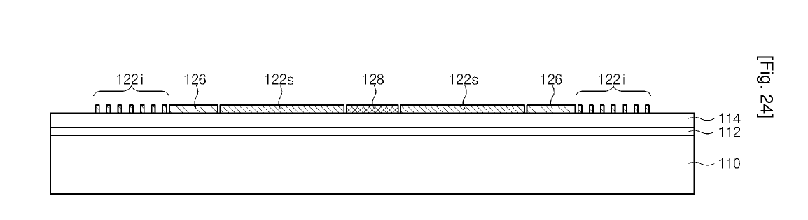

FIGS. 15 through 24 are cross-sectional views taken along line I-I′ of FIG. 1, illustrating a method of fabricating a bio lab-on-a-chip according to an embodiment of the present invention.

*Referring to FIGS. 15 and 16, a substrate 110 is provided. The substrate 110 may include at least one selected from silicon, glass, plastic, metal, and a combination thereof. Preferably, the substrate 100 may be a silicon substrate.

The silicon oxide (SiO₂) film 112 may be formed on the substrate 110. The SiO₂ film 112 may be provided for minimizing the loss of a surface acoustic wave (SAW), which should propagate along a piezoelectric thin film 114, by preventing the SAW from propagating to the substrate 110.

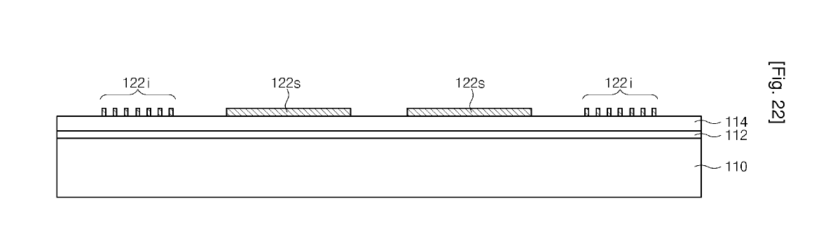

Referring to FIG. 17, the piezoelectric thin film 114 may be formed on the SiO₂ film 112. The piezoelectric thin film 114 may be formed to have a thickness in the range of about 0.1 μm to about 10 μm. Preferably, the piezoelectric thin film 114 may be formed to have a thickness in the range of about 0.5 μm to about 10 μm.

The step of forming the piezoelectric thin film 114 may include the step of depositing a piezoelectric material on the substrate 110 and the step of heat-treating the deposited piezoelectric material. The piezoelectric material may include at least one selected from ZnO, AlN, LiNbO₃, LiTaO₃, quartz, polymer, and a combination thereof. The step of depositing the piezoelectric material may include at least one method selected from a reactive sputtering method, a chemical vapor deposition (CVD) method, a molecular beam epitaxy method, an atomic layer deposition (ALD) method, and a combination thereof. Preferably, the piezoelectric thin film 114 may be a film which is heat-treated at about 400° C. under N₂ atmosphere for about 10 minutes after ZnO is deposited in a thickness of about 5.5 μm by the reactive sputtering method. The deposition method for the piezoelectric thin film 114 may be for the decrease of stresses applied on the deposited piezoelectric material and the enhancement of the crystallinity of the piezoelectric thin film 114.

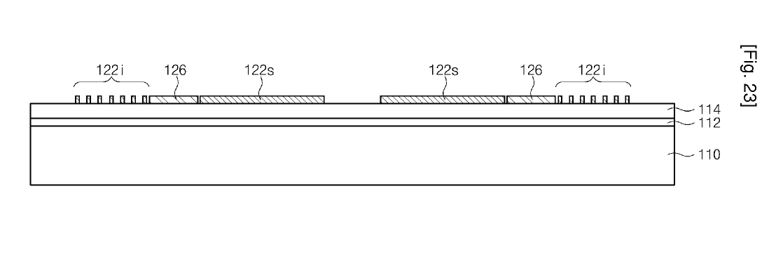

Referring to FIG. 18 through 20, a photoresist 116 may be applied on the piezoelectric thin film 114. A mask pattern 118 may be provided on the photoresist 116. By performing a photo etching process using the mask pattern 118 as a mask, a photoresist pattern 116a may be formed to expose a fluidic controller region A (including a sensing interdigitated transducer region) and a sensor region B on the piezoelectric thin film 114.

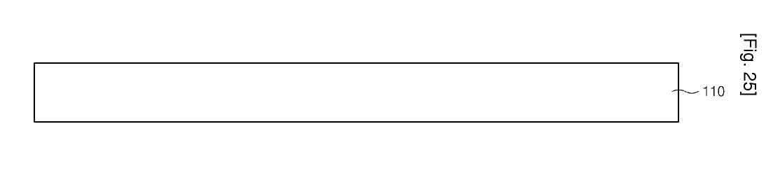

Referring to FIGS. 21 and 22, after the mask pattern 118 is removed, a conductive metal film 120 may be formed on the photoresist pattern 116a and on the piezoelectric thin film 114 exposed by the photoresist pattern 116a. The conductive metal film 120 may include at least one selected from Au, Ag, Al, Pt, W, Ni, Cu, and a combination thereof.

The photoresist pattern 116a, and the conductive metal film 120 on the photoresist pattern 116a may be removed by a lift-off process. Accordingly, a sensor 122s and a fluidic controller 122i (including a sensing interdigitated transducer) may be formed on the piezoelectric thin film 114. The fluidic controller 122i may have a form of an interdigitated transducer.

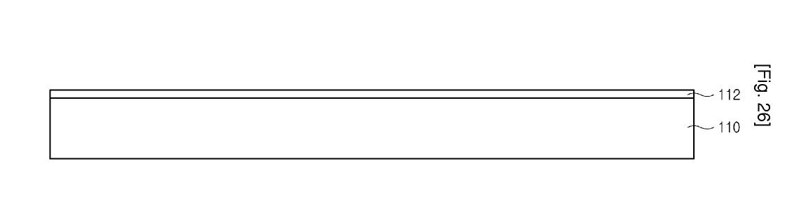

Referring to FIG. 23, a microfluidic channel 126 may be formed on the piezoelectric thin film between the sensor 122s and the fluidic controller 122i. The microfluidic channel 126 may be formed as a hydrophobic material. The hydrophobic material may include at least one material selected from a silane compound, a carbon nanotube (CNT), and a diamond like carbon (DLC). Accordingly, a microfluidic in the form of a liquid drop may be transferred to the sensor 122s through the microfluidic channel 126 while maintaining its form.

Though not shown, the formation of antibodies (See 124 in FIG. 2) on the sensor 122s may be further included. The antibodies may include a self-assembling monolayer (SAM) or protein.

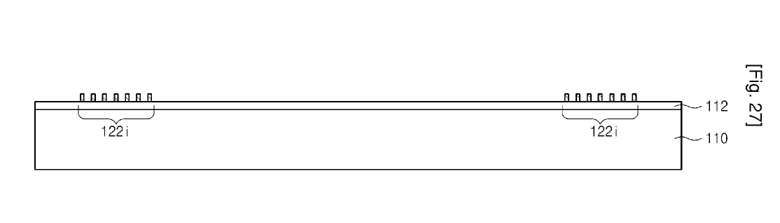

Referring to FIG. 24, a dam portion 128 which surrounds the sensor 122s and the microfluidic channel 126 may be formed. The dam portion 128 may be formed as a photosensitive polymer. Accordingly, the microfluidic in the form of a liquid drop may be stably transferred to the sensor 122s through the microfluidic channel 126 without deviating outside.

FIGS. 25 through 31 are cross-sectional views taken along line I-I′ of FIG. 1, illustrating a method of fabricating a bio lab-on-a-chip according to another embodiment of the present invention.

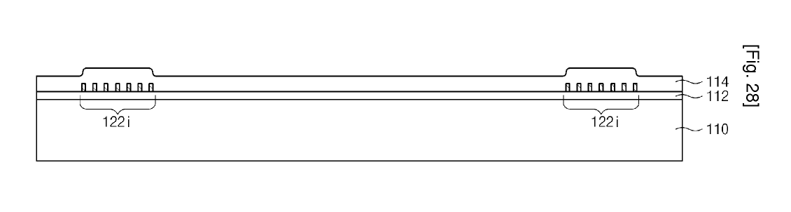

Referring to FIGS. 25 and 26, a substrate 110 is prepared. The substrate 110 may include at least one selected from silicon, glass, plastic, metal, and a combination thereof. Preferably, the substrate 110 may be a silicon substrate.

The silicon oxide (SiO₂) film 112 may be formed on the substrate 110. The SiO₂ film 112 may be provided for minimizing the loss of a surface acoustic wave (SAW), which should propagate along the piezoelectric thin film 114, by preventing the SAW from propagating to the substrate 110.

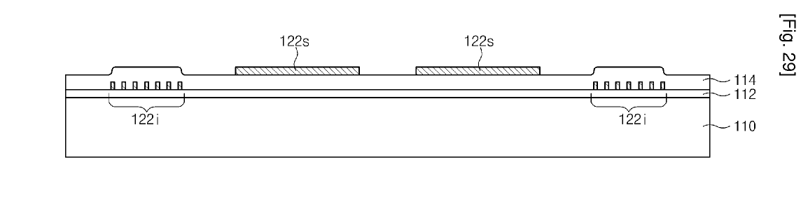

Referring to FIGS. 27 and 28, a fluidic controller 122i (including a sensing interdigitated transducer) may be formed on the SiO₂ film 112. The fluidic controller 122i may include at least one selected from Au, Ag, Al, Pt, W, Ni, Cu, and a combination thereof. The fluidic controller 122i may have a form of an interdigitated transducer.

The piezoelectric thin film 114 may be formed to cover the fluidic controller 122i on the SiO₂ film 112. The piezoelectric thin film 114 may be formed to have a thickness in the range of about 0.1 μm to about 10 μm. Preferably, the piezoelectric thin film 114 may be formed to have a thickness in the range of about 0.5 μm to about 10 μm.

The step of forming the piezoelectric thin film 114 may include the step of depositing a piezoelectric material on the substrate 110 and the step of heat-treating the deposited piezoelectric material. The piezoelectric material may include at least one selected from ZnO, AlN, LiNbO₃, LiTaO₃, quartz, polymer, and a combination thereof. The step of depositing the piezoelectric material may include at least one method selected from a reactive sputtering method, a CVD method, a molecular beam epitaxy method, an atomic layer deposition (ALD) method, and a combination thereof. Preferably, the piezoelectric thin film 114 may be a film which is heat-treated at about 400° C. under N₂ atmosphere for about 10 minutes after ZnO is deposited in a thickness of about 5.5 μm by the reactive sputtering method. The deposition method for the piezoelectric thin film 114 may be for the decrease of stresses applied on the deposited piezoelectric material and the enhancement of the crystallinity of the piezoelectric thin film 114.

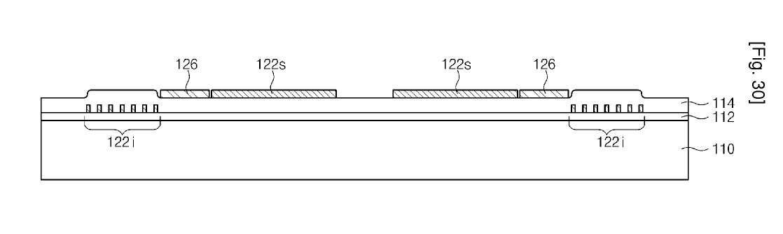

Referring to FIG. 29, a sensor 122s may be formed on the piezoelectric thin film 114. The sensor 122s may include at least one selected from Au, Ag, Al, Pt, W, Ni, Cu, and a combination thereof.

Though not shown, the formation of antibodies (See 124 in FIG. 2) on the sensor 122s may be further included. The antibodies may include a self-assembling monolayer (SAM) or protein.

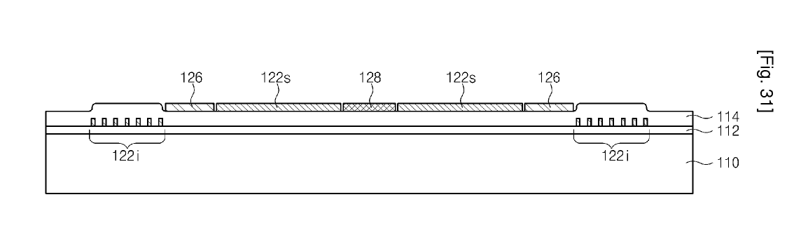

Referring to FIG. 30, a microfluidic channel 126 may be formed on the piezoelectric thin film 114 between the sensor 122s and the fluidic controller 122i. The microfluidic channel 126 may be formed as a hydrophobic material. The hydrophobic material may include at least one material selected from a silane compound, a carbon nanotube (CNT), and a diamond like carbon (DLC). Accordingly, a microfluid in the form of a liquid drop may be transferred to the sensor 122s through the microfluidic channel 126 while maintaining its form.

Referring to FIG. 31, a dam portion 128 which surrounds the sensor 122s and the microfluidic channel 126 may be formed. The dam portion 128 may be formed as a photosensitive polymer. Accordingly, the microfluid in the form of a liquid drop may be stably transferred to the sensor 122s through the microfluidic channel 126.

Since a bio lab-on-a-chip according to the methods of fabricating the same described above may perform transferring, stopping, reacting, and sensing of a microfluid in the form of a nanoliter volume drop solution, all the processes of the chemical analysis may be performed on a single chip while using the minimum volume of a sample. Accordingly, the costs of analysis may be lowered simultaneously with the reduced fabricating costs of a bio lab-on-a-chip.

Since a bio lab-on-a-chip according to embodiments of the present invention described above may perform transferring, stopping, reacting, and sensing of a microfluid in the form of a nanoliter volume drop solution, the minimization of the consumption of a bio sample and reagents may be achieved. Accordingly, the costs of analysis may be lowered. Further, since all the processes of the chemical analysis are performed on a single chip, a rapid and exact analysis may be made. In addition, the reduction of the fabricating costs by replacing an expensive bulk substrate with a piezoelectric thin film. Additionally, since the present invention may be applied to a multi-use semiconductor manufacturing process, it may be applicable to various bio lab-on-a-chip fields including a protein lab-on-a-chip, a polymerase chain reaction (PCR) chip, deoxyribonucleic acid (DNA) lab-on-a-chip or a micro biological/chemical reactor.

### INDUSTRIAL APPLICABILITY

The present invention may apply to a bio-micro electronic mechanical systems (bio-MEMS) for chemical analysis of bio samples and instrumentation of bio signals.

1. A bio lab-on-a-chip comprising:
a substrate;
a piezoelectric thin film on the substrate;
a sensing unit provided on the piezoelectric thin film, and sensing a bio signal of a microfluid; and
a fluidic control unit adjacent to the sensing unit, and controlling a transfer of the microfluid.

2. The bio lab-on-a-chip of claim 1, further comprising a microfluidic channel disposed on the piezoelectric thin film between the sensing unit and the fluidic control unit.

3. The bio lab-on-a-chip of claim 2, wherein the microfluidic channel comprises a hydrophobic material.

4. (canceled)

5. The bio lab-on-a-chip of claim 1, wherein the substrate comprises at least one selected from silicon, glass, plastic, metal, and a combination thereof.

6. The bio lab-on-a-chip of claim 1, wherein the piezoelectric thin film has a thickness in the range of about 0.1 μm to about 10 μm.

7. (canceled)

8. The bio lab-on-a-chip of claim 1, further comprising antibodies provided on the sensing unit.

9. The bio lab-on-a-chip of claim 8, wherein the antibodies comprise a self-assembling monolayer (SAM) or protein.

10. The bio lab-on-a-chip of claim 1, further comprising a pair of interdigitated transducers disposed adjacent to the sensing unit in a vertical direction to a virtual line connecting the fluidic control unit and the sensing unit, wherein the sensing unit is positioned between the pair of interdigitated transducers.

11. The bio lab-on-a-chip of claim 10, wherein the pair of interdigitated transducers comprise:
a selected interdigitated transducer sending a surface acoustic wave (SAW) to the sensing unit; and
an unselected interdigitated transducer converting a modulated SAW by the sensing unit into an electrical signal.

12. The bio lab-on-a-chip of claim 1, wherein the fluidic control unit is an interdigitated transducer which provides a SAW in a direction to the sensing unit.

13. The bio lab-on-a-chip of claim 1, further comprising a dam portion which surrounds the sensing unit and the microfluidic channel.

14-30. (canceled)

31. A method of operating a bio lab-on-a-chip, the method comprising:
providing a microfluid to a region between a sensing unit and a fluidic control unit adjacent to each other on a substrate having a piezoelectric material;
transferring the microfluid to the sensing unit using a surface acoustic wave (SAW) generated by driving the fluidic control unit; and
sensing a bio signal of the microfluid at the sensing unit.

32. The method of claim 31, wherein the fluidic control unit is an interdigitated transducer for fluid control, which provides the SAW.

33. The method of claim 31, wherein the microfluid is a liquid drop of nanoliters in volume.

34. The method of claim 31, wherein the microfluid comprises one of an optical marker material and a radioactive marker material.

35. The method of claim 31, wherein the sensing of the bio signal of the microfluid comprises sensing a reaction between antibodies provided on the sensing unit and the microfluid as an optical signal or a radioactive signal.

36. The method of claim 31, wherein the sensing of the bio signal of the microfluid comprises sensing a reaction between antibodies provided on the sensing unit and the microfluid as an electrical signal.

37. The method of claim 36, wherein the sensing of the electrical signal uses at least one interdigitated transducer disposed adjacent to the sensing unit, and measures a resonance frequency modulated as an SAW generated from the interdigitated transducer passes through the sensing unit.

38. The method of claim 37, wherein a variation of the resonance frequency of the SAW may be proportional to the amount of a reaction between the antibodies and the microfluid.

39. The method of claim 37, wherein the interdigitated transducer comprises:
a first detection interdigitated transducer sending the SAW to the sensing unit; and
a second detection interdigitated transducer detecting the modulated SAW at the sensing unit.

40-42. (canceled)
